(19) United States
(12) Patent Application Publication
(10) Pub. No.: US 2021/0185159 A1
(43) Pub. Date: Jun. 17, 2021

(54) MULTI-FUNCTION MOBILE PHONE CASE WIRELESS CHARGER, VIRTUAL DIETITIAN ELECTRONIC WEIGHING DEVICE

(71) Applicants: Shenzhen Zhiaijiaju Keji Youxian Gongsi, Shenzhen (CN); Edgar Alejandro Olvera Rojas, Puebla (MX)

(72) Inventor: Zeng Hongxing, Xian (CN)

(21) Appl. No.: 16/709,980

(22) Filed: Dec. 11, 2019

(51) Int. Cl. H04M 1/21; H04B 1/3888; H02J 7/00; H05K 5/00; G01G 19/52; G09B 19/00; G09B 5/02; G16H 20/60

(52) U.S. Cl. CPC H04M 1/21; H04B 1/3888; H02J 7/0044; G16H 20/60; G01G 19/52; G09B 19/0092; G09B 5/02; H05K 5/0017

(57) ABSTRACT

A plurality of electronic scale sensors are fixed on one side of the top end of the casing, and the top ends of the plurality of electronic scale sensors are respectively called stainless steel. The four corners of the bottom end of the disc are fixedly connected. The utility model can realize placing the object on the electronic scale shell by setting the electronic scale sensor, the stainless steel weighing disc and the PCB board display, and the electronic scale sensor transmits the weighed weight. To the PCB display screen, and display the specific value through the PCB board display, so that the mobile phone case has the function of electronic scale, and the information on the PCB board display can be transmitted to the mobile phone by connecting the PCB board with the mobile phone by Bluetooth or any other connection way available. Individualized nutrition programs can be customized for the user, including calories, protein, carbohydrates, fats, etc., and each food they eat can be tracked through an integrated weighing device.

### FIELD OF THE INVENTION

The utility model relates to a mobile phone shell, in particular to an electronic weighing device with wireless charging function, and a virtual dietitian of a multifunctional mobile phone shell, belonging to the technical field of daily necessities.

### BACKGROUND OF THE INVENTION

The phone case is an ornament that protects or decorates the appearance of the phone. With the rapid development of science and technology, the industry of science and technology is emerging as a new type of industry. Fashion IT brands are diversified with the market. Diversified for the increase of mobile phone brand and function, the mobile phone protective case is divided into PC shell, leather, silicone, cloth, hard plastic, leather case, metal tempered glass case, soft plastic, cashmere, silk and so on. The mobile phone case not only serves as an ornament to make your mobile phone a landscape, but also protects the mobile phone from falling, scratching, waterproof and shockproof. In the prior art, there are many common mobile phone cases, electronic scales or weighing devices, mobile phone chargers, metals and plastics or any other materials on the market, but all exhibit a single product style, designed according to traditional purposes, and the mobile phone case The function is relatively simple, while the modern people's diet is generally irregular, and mobile phones are the most commonly used tools, so you can use mobile phones to help people develop healthy eating habits.

### SUMMARY OF THE INVENTION

The purpose of the utility model is to provide a multifunctional mobile phone shell charging virtual dietitian electronic weighing device to solve the problem that the mobile phone shell has a single function in the above background art. In order to achieve the above object, the present invention provides the following technical solution: a multifunctional mobile phone case wireless charger, virtual dietitian electronic weighing device, comprising a housing, and a sensor bracket is fixedly mounted on one side of the top end of the housing, the sensor bracket The top end is fixedly provided with a plurality of electronic scale sensors, and the top ends of the plurality of electronic scale sensors are respectively fixedly connected to the four corners of the bottom end of the stainless steel weighing pan.

As a preferred technical solution of the present invention, a middle portion of the top end of the housing is fixedly provided with a PCB display screen, and the PCB display screen is electrically connected to the electronic scale sensor.

As a preferred technical solution of the present invention, a plastic cover is connected to the top end of the PCB display screen, and an opening is formed in a middle portion of the top end of the plastic cover, and a transparent plastic sheet is embedded in the interior of the opening.

As a preferred technical solution of the present invention, the top end of the plastic cover is attached with a TPE sticker, and the shape of the TPE sticker matches the shape of the plastic cover.

As a preferred technical solution of the present invention, a battery is fixedly disposed on the other side of the top end of the casing, and a charging plug is fixed in a middle portion of one end of the battery, and the battery is electrically connected to the PCB display screen and the mobile phone.

As a preferred technical solution of the present invention, the top end of the housing is provided with a plurality of slots, the slots are engaged with the corresponding plungers, and the plurality of the plungers are respectively fixedly disposed on the electronic scale housing. At the bottom end the bottom end of the electronic scale housing is provided with a viewing port.

Compared with the prior art, the beneficial effects of the utility model are:

1. The utility model relates to a multifunctional mobile phone case wireless charger, virtual dietitian electronic weighing device, which can realize placing an object on an electronic scale shell by setting an electronic scale sensor, a stainless steel weighing plate (or any other proper material) and a PCB board display, and the electronic scale sensor will transmit to the PCB display screen the weighed weight, and the specific value is displayed through the PCB board display screen, so that the mobile phone case has the function of electronic scale;

2. The utility model relates to a multifunctional mobile phone shell charging virtual dietitian electronic weighing device, which can realize the information transmission on the PCB board display to the mobile phone by connecting the PCB board display screen with the mobile phone by any way of data transfer, and customize the personalized for the user. Nutritional programs, including calories, protein, carbohydrates, fats, etc., can track every food they eat through integrated weighing equipment. Users can also customize healthy recipes and even order the ingredients, or the food they need to help them develop healthy Eating habits.

### DESCRIPTION OF THE DRAWINGS

FIG. 1 is a schematic view of the structure of the present invention;

FIG. 2 is a schematic view showing the internal structure of the casing of the present invention;

FIG. 3 is a schematic view showing the internal structure of the electronic scale housing of the present invention;

4 is a schematic structural view of an electronic sensor of the present invention;

FIG. 5 is a schematic view showing the structure of the plastic cover of the present invention.

In the figure: 1, housing; 2, electronic scale sensor; 3, stainless steel weighing plate or any other viable material; 4, electronic scale shell; 5, TPE sticker; 6, plastic cover; 7, battery; 8, charging plug; 9, PCB board display Screen; 10, slot; 11, plunger; 12, opening; 13, sensor bracket.

### DETAILED DESCRIPTION

The technical solutions in the embodiments of the present invention will be clearly and completely described in conjunction with the drawings in the embodiments of the present invention. It is obvious that the described embodiments are only a part of the embodiments of the present invention, and not all of the embodiments. example. All other embodiments obtained by those skilled in the art based on the embodiments of the present invention without creative efforts are within the scope of the present invention.

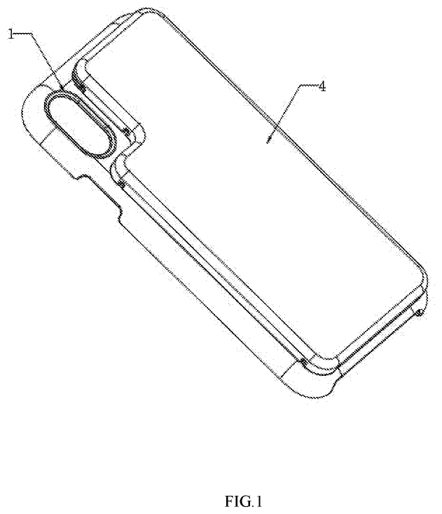

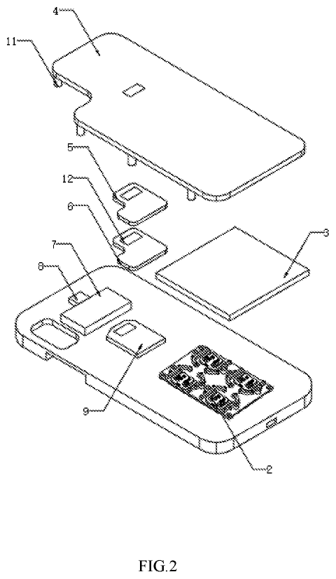

Referring to FIG. 1-5, the utility model provides a technical solution for a multifunctional mobile phone shell charging virtual dietitian electronic weighing device:

According to FIG. 1 to FIG. 5, the housing 1 is included, and the sensor holder 13 is fixedly mounted on one side of the top end of the housing 1. The top of the sensor holder 13 is fixedly provided with a plurality of electronic scale sensors 2, and a plurality of electronic scale sensors 2 The top end is fixedly connected to the four corners of the bottom end of the stainless steel weighing plate 3 respectively, and the middle part of the top end of the housing 1 is fixedly provided with a PCB board display 9, and the PCB board display screen 9 is electrically connected with the electronic scale sensor 2, and the electronic scale sensor 2 The pressure change on the stainless steel scale 3 can be sensed, and the weighed weight is transmitted to the PCB display 9, and the specific value is displayed through the PCB display 9.

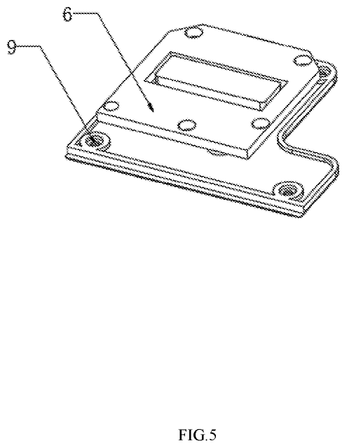

According to FIG. 2, FIG. 4 and FIG. 5, the top end of the PCB board display 9 is connected with a plastic cover 6, and the plastic cover 6 is provided for waterproofing. The opening of the top end of the plastic cover 6 is provided with an opening 12, A transparent plastic sheet is embedded in the interior of the opening 12, and the transparent plastic sheet does not affect the observation reading while being waterproof. The top end of the plastic cover 6 is attached with a TPE sticker 5, and the TPE sticker 5 provided has a high temperature resistance to prevent temperature. Excessively affecting the work of the PCB board display 9, the shape of the TPE sticker 5 is matched with the shape of the plastic cover 6, the battery 7 is fixedly disposed on the other side of the top end of the housing 1, and the charging plug 8 is fixed in the middle of the battery 7 at one end. The battery 7 is electrically connected with the PCB display 9 and the mobile phone. The battery 7 can charge the mobile phone, and the power is increased for the mobile phone in an emergency situation, and the information on the PCB display 9 is transmitted to the mobile phone to customize the personality for the user. Nutritional programs, including calories, protein, carbohydrates, fats, etc., can be used to track every food they eat through integrated weighing equipment. Users can also customize healthy recipes and even order the ingredients or the food they need.

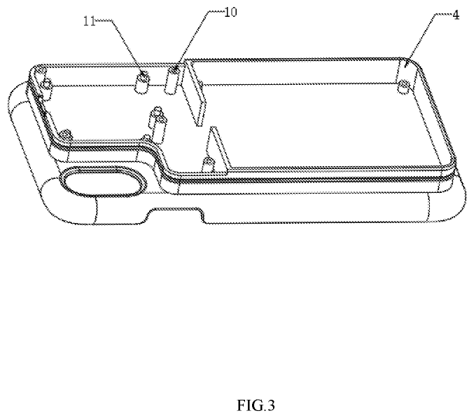

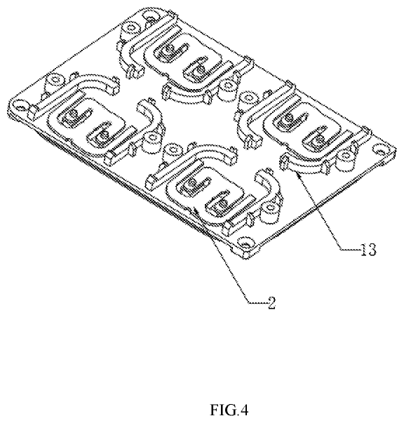

According to FIG. 3, a plurality of slots 10 are defined in the top end of the housing 1, and the slot 10 is engaged with the corresponding plunger 11, and the slot 10 and the plug 11 are provided to facilitate the installation of the electronic housing 4. And disassembling, convenient for maintenance and maintenance, a plurality of plungers 11 are respectively fixedly disposed at the bottom end of the electronic scale housing 4, and the bottom end of the electronic scale housing 4 is provided with a viewing port, and the PCB board display screen can be directly observed through the observation port. The reading on it.

In specific use, the utility model relates to a multifunctional mobile phone case wireless charger, virtual dietitian electronic weighing device, which firstly places an object on the electronic scale housing 4, and the electronic scale sensor 2 can sense the pressure change on the stainless steel weighing plate 3, and The weight data is transferred to the PCB display screen 9, and the specific value is displayed through the PCB display 9, and the information on the PCB display 9 is transmitted to the mobile phone to customize a personalized nutrition program for the user. Including calories, protein, carbohydrates, fats, etc., and through the integrated weighing equipment to track every food they eat, users can also customize healthy recipes, and even order the required ingredients, when the battery 7 is exhausted, Insert the charging cable into the charging plug 8 to charge the battery 7. In an emergency, the battery 7 can charge the mobile phone. When the maintenance is needed, the electronic housing 4 is removed and the inside is inspected. After the repair is completed, the battery will be inserted 11 is inserted into the interior of the corresponding slot 10, and is pressed hard to close the electronic scale housing 4.

In the description of the present invention, it is to be understood that the orientation or positional relationship of the indications is based on the orientation or positional relationship shown in the drawings, and is merely for convenience of description of the present invention and simplified description, rather than indicating or implying The device or component must have a particular orientation, configuration and operation in a particular orientation, and thus is not to be construed as limiting the invention.

In the present invention, unless explicitly defined and defined, for example, it may be a fixed connection, a detachable connection, or an integral; it may be a mechanical connection or an electrical connection; it may be directly connected, It can be indirectly connected through an intermediate medium, and can be an internal connection between two elements or an interaction relationship between two elements. Unless otherwise explicitly defined, those skilled in the art can understand the above terms according to specific circumstances. The specific meaning in the utility model.

While the embodiments of the present invention have been shown and described, it will be understood by those skilled in the art The scope of the invention is defined by the appended claims and their equivalents.

What is claimed is:

1. A multifunctional mobile phone case wireless charger, virtual dietitian electronic weighing device, comprising a housing (1), characterized in that: a sensor holder (13) is fixedly mounted on one side of a top end of the housing (1);
a PCB display screen (9) is mounted on the top end of the housing (1);
a plurality of electronic scale sensors (2) are fixed to the top end of the sensor holder (13); and the top ends of the plurality of electronic scale sensors (2) are fixedly connected to the four corners of the bottom end of the stainless steel weighing disc (3), respectively.

2. The multi-function mobile phone case wireless charger, virtual dietitian electronic weighing device according to claim 1, wherein the PCB board display (9) is electrically connected to the electronic scale sensor (2).

3. The multi-function mobile phone case wireless charger, virtual dietitian electronic weighing device according to claim 1, wherein the top end of the PCB display screen (9) is connected with a plastic cover (6); an opening (12) is defined in a middle portion of the top end of the plastic cover (6); and a transparent plastic sheet is embedded in the interior of the opening (12).

4. The multi-function mobile phone shell wireless charger, virtual dietitian electronic weighing device according to claim 3, wherein the top end of the plastic cover (6) is attached with a TPE sticker (5) such that the shape of the TPE sticker (5) matches the shape of the plastic cover (6).

5. The multifunctional weighing device charging virtual dietitian electronic weighing device according to claim 1, wherein the other side of the top end of the casing (1) is fixedly provided with a battery (7); a charging plug (8) is fixed in a middle portion of one end of the battery (7); and the battery (7) is electrically connected to the PCB display screen (9) and the mobile phone.

6. The multifunctional virtual mobile phone shell charging virtual dietitian electronic weighing device according to claim 1, wherein the top end of the housing (1) is provided with a plurality of slots (10); the slot (10) is engaged with a corresponding plunger (11); and the plurality of the plungers (11) are respectively fixedly disposed at the bottom end of the electronic scale housing (4), wherein the bottom end of the electronic scale housing (4) Open an observation port.
